(19) United States
(12) Patent Application Publication
(10) Pub. No.: US 2026/0234293 A1
(43) Pub. Date: Aug. 13, 2026

(54) PURIFICATION OF CAPSULAR POLYSACCHARIDES

(71) Applicant: Vaxcyte, Inc., San Carlos, CA (US)

(72) Inventors: Peter T. Davey, Foster City, CA (US); Marc Wong, San Mateo, CA (US); Kathleen Ma, San Francisco, CA (US); Brian Fennell, Goleta, CA (US)

(21) Appl. No.: 19/471,043

(22) PCT Filed: Apr. 3, 2024
(86) PCT No.: PCT/US2024/022744

§ 371 Date: Sep. 30, 2025

Related U.S. Application Data
(60) Provisional application No. 63/457,632, filed on Apr. 6, 2023.

(51) Int. Cl. C08B 37/00; B01D 15/38; B01D 39/20; B01D 61/14; B01D 71/02; B01D 71/68

(52) U.S. Cl. CPC C08B 37/0003; B01D 15/38; B01D 39/2055; B01D 61/146; B01D 71/022; B01D 71/68; B01D 2257/91

(57) ABSTRACT

Described herein are methods of purifying various S. pneumoniae serotype capsular polysaccharides from cell lysates, which improve the yield of polysaccharides while minimizing the amount of Host Cell Protein (HCP) in the product.

### CROSS REFERENCE TO RELATED APPLICATIONS

This application claims priority to U.S. Patent Application No. 63/457,632, filed on Apr. 6, 2023, the contents of which are incorporated herein in their entirety.

### TECHNICAL FIELD

This invention relates generally to processes of purifying polysaccharides, specifically purifying capsular polysaccharides for improved overall yield.

### BACKGROUND

Streptococcus pneumoniae causes a significant number of invasive infections in both infants and older adults. Polyconjugate vaccines have been successful in attenuating the effects of such bacterial infections. One major challenge associated with polyconjugate vaccines is the number of polysaccharides to be prepared for a single dose of vaccine. Current and emerging Pneumococcal conjugate vaccines contain twenty-plus serotypes of the S. pneumoniae serotypes, presenting a massive manufacturing undertaking. One major challenge to producing conjugate vaccines is that certain serotypes have consistently generated much lower amounts of polysaccharide. If the polysaccharide yield of these serotypes is improved, manufacturers could save millions of dollars a year in producing conjugate vaccines.

Thus, there is a need for improved polysaccharide yield for the preparation of polyconjugate vaccines against bacterial infections, such as S. pneumoniae.

### BRIEF DESCRIPTION OF THE DRAWINGS

FIG. 1 shows a schematic of an overall purification of polysaccharide set-up.

FIG. 2 is a flow diagram showing excerpted steps of capsular polysaccharide lysate purification.

FIG. 3A shows polysaccharide yield and HCP clearance after a carbon filter step in an overall capsular polysaccharide purification scheme.

FIG. 3B shows polysaccharide yield and HCP clearance after a ceramic hydroxyapatite (CHT) chromatography step of an overall capsular polysaccharide purification scheme.

FIG. 4A is a bar graph showing S. pneumoniae serotype 18C yields for various batches.

FIG. 4B is a bar graph showing host cell protein (HCP) levels in various S. pneumoniae serotype 18C manufacturing batches.

FIG. 4C is a bar graph showing polysaccharide product yields using standard carbon filter usage to according to carbon filter specifications.

FIG. 5A is a bar graph showing polysaccharide product yields using standard purification steps relative to the optimized processes described herein.

FIG. 5B is a bar graph showing HCP (host cell protein) levels in S. pneumoniae 18C purification using the improved polysaccharide purification process.

FIG. 6A is a bar graph showing carbon filter throughput versus yield at different load capacities for S. pneumoniae serotypes 5, 14, and 18C.

FIG. 6B is a bar graph showing carbon filter HCP clearance for S. pneumoniae serotypes 5, 14, and 18C at different load capacities.

FIG. 7 is a bar graph showing HCP (host cell protein) reduction from a carbon filter step relative to a ceramic hydroxyapatite (CHT) column step for S. pneumoniae serotypes 5, 14, and 18C.

### DETAILED DESCRIPTION

Provided herein are processes for purifying capsular polysaccharides from cell lysates, which improve the yield of capsular polysaccharides while minimizing the amount of Host Cell Protein (HCP) in the product.

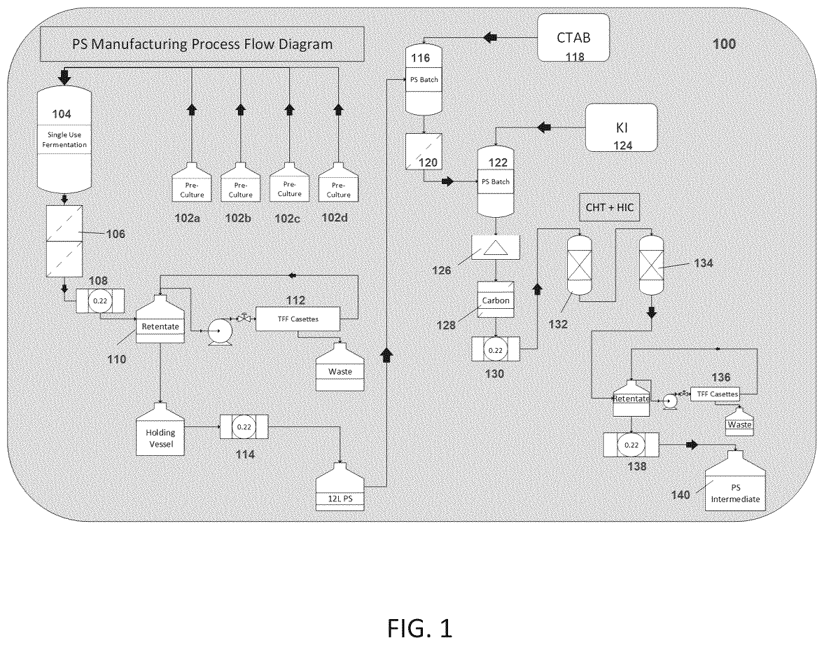

The purification of capsular polysaccharide cell lysates involves multiple steps of filtration and column chromatography. FIG. 1 is an overall diagram of an exemplary polysaccharide manufacturing process flow diagram process 100. FIG. 1 is a non-limiting example of one possible arrangement for purifying capsular polysaccharide cell lysates. While some desired polysaccharide product is lost at each step of purification, a large amount is typically lost at the carbon filter process step, which retains anywhere from 20-30% of the product, whereas the majority of the other steps in the purification process provide yields of greater than 60% product recovery. The carbon filter step importantly helps to remove host cell protein (HCP) that the downstream purification steps (e.g., CHT column, hydrophobic interaction chromatography (HIC) and final tangential flow filtration (TFF) steps) cannot remove. The amount of HCP that is present in the final polysaccharide sample solution should be less than 11%.

The capsular polysaccharide purification processes provided herein, comprise, inter alia, a carbon filtering step where the sample amount flowed through the carbon filter is about three times the amount suggested for the carbon filter area. Additionally, ceramic hydroxyapatite chromatography is run at about half the load capacity to remove excess host cell proteins (HCP) from the prior purification steps. Thus, the capsular polysaccharide purification processes provided herein increase polysaccharide yield while minimizing the amount of HCP present.

Process 100 starts with precultures 102a-102d in one or more containers that are introduced to a fermentation reactor 104 where fermentation of various capsular polysaccharides occurs. Once the fermentation process is completed as generally known the art (e.g., L. B. Holt, The Culture of Streptococcus pneumoniae, J. gen. Microbiol., 1962, 27, pg. 327-330; Lee et. Al. Quality Improvement of Capsular Polysaccharide in Streptococcus pneumoniae by Purification Optimization, Front. Bioeng. Biotechnol., Feb. 4, 2020, Vol. 8, Article 39), the resulting cells are lysed using standard lysing procedures (e.g., 106 in FIG. 1). The resulting crude lysate is then introduced into the purification process flow. The term ‘crude’ is used to refer to a solution post cell lysis that includes the product polysaccharide of interest along with cellular debris that is to be removed.

Referring to FIG. 1, a capsular polysaccharide cell lysate is introduced into one or more depth filters 108. If there is more than one depth filter, the individual depth filters may be placed in series. In some embodiments, depth filters 108 may comprise a matrix of cellulose and diatomaceous earth. In some embodiments, a first depth filters 108 may have asymmetric nominal pore sizes of about 10 to 0.5 micrometers while a second depth filter 108, in series with the first may have an asymmetric nominal pore size rating of 2 to 0.2 micrometers. In some embodiments, depth filters 108 may be flushed with a solution of 25 mM sodium phosphate buffer and 444 mM NaCl where the pH is approximately 6.8 (Buffer J). Buffer J may be used to aid in the filtration of the capsular polysaccharide cell lysate through the one or more depth filters 108 to produce a depth-filtered polysaccharide. In some embodiments, depth filters 108 may be flushed with water followed by buffer prior to use. In some embodiments, the filtration flow rate for sending the capsular polysaccharide cell lysate through depth filters 108 is about 10 L/min. In some embodiments, the temperature for filtration using depth filters 108 is about 18° C. and 22° C. In some embodiments, the filtration pressure does not exceed about 2 bars. In some embodiments, the pH is maintained at about 6.6 to about 7.

In some instances, with reference to FIG. 1, prior to introducing the depth filtered polysaccharide into a tangential flow filtration (TFF) system 112, it can be run through a subsequent filter to remove excess bioburden. As a non-limiting embodiments, a 0.22 micron filter (e.g., Sartropore platinum) or the like may be used. In some embodiments, the flow rate through the 0.22 micron filter is between about 9 L/min to about 11 L/min. The 0.22 micron filter may be pre-flushed with NaOH, followed by water. In some instances, the 0.22 micron filter can be flushed with 25 mM sodium phosphate buffer and 444 mM NaCl pH 6.8 post filtration.

The depth-filtered polysaccharide is then introduced into the TFF system 112. As a non-limiting example, TFF system 112 may be composed of polyethersulfone membrane. In certain embodiments, TFF system 112 may include polypropylene screen material. In some embodiments, TFF system 112 may be performed at a molecular weight cut-off (MWCO) of about 100 kDa. TFF system 112 may be equilibrated with a buffer containing NaCl (Buffer A) and Buffer A is used to flow the depth-filtered polysaccharide through TFF system 112 where a TFF-purified polysaccharide is retained in vessel 110a and holding vessel 110b. Additional Buffer A may be flushed through TFF system 112. The TFF-purified polysaccharide may also be further filtered (e.g., 0.2 microns) with Buffer A as a flush with filter 114.

Some embodiments of TFF system 112 system can be an ultrafiltration/diafiltration arrangement. In some embodiments, for a 300 kg volume containing the depth-filter polysaccharide, the 300 kg volume can be concentrated to about 40 kg (ultrafiltration) with a circulation time of about 15 minutes. The 40 kg concentrated polysaccharide sample is run through a diafiltration process with about ten times the weight of a circulating buffer solution of sodium phosphate buffer and sodium chloride (Buffer J) where the pH is neutral. In some embodiments, Buffer J is about 25 mM Na3PO4, about pH 6.8 and about 444 mM NaCl (e.g., such as buffers disclosed in WO2021108792). As mentioned above, the molecular weight cutoff (MWCO) is about 100 kDa. In some embodiments the collection vessel may be agitated between about 90-110 rpms in order to keep the solution homogeneous. In some embodiments, the TFF system 112 step is performed at a temperature between 15° C. and 25° C. In some embodiments the cross-flow rate is between about 1500 L/hr to about 1700 L/hr. In some embodiments, the ultrafiltration is performed at a pressure of between about 0.35 and about 0.75 bars and diafiltration is performed at a pressure of between about 0.80 and about 1.20 bars. The resulting TFF polysaccharide can be concentrated prior to going to the next step.

Following TFF 112, the TFF-purified polysaccharide in batch container 116 may be precipitated with cetyltrimethylammonium bromide (CTAB) 118. In some embodiments, 1.0±0.05% CTAB where the pH is adjusted to approximately 6.8 may be used. In addition, the CTAB precipitation may be over a 60-75 minute period at a temperature of 30±3° C. to precipitate out impurities resulting in a CTAB precipitated polysaccharide that is in the supernatant (e.g., in solution). CTAB impurities may then be filtered with an additional depth filter where the depth filter is comprised of cellulose and diatomaceous earth. In some embodiments, this depth filter has a nominal pore size range of approximately 40 to 0.6 microns. In some embodiments after filtering, the depth filter may be flushed with a solution of 20 mM sodium phosphate buffer, 400 mM NaCl, and 15 CTAB at pH 6.8 (Buffer B).

In some instances, the CTAB precipitated polysaccharide solution can be run again through a depth filter for removing additional bioburden present. In some embodiments, the depth filter is a 40 micron filter.

Following CTAB precipitation, a second precipitation step using potassium iodide (KI) 124 may be applied to the CTAB-precipitated polysaccharide at 122 to obtain a KI-precipitated polysaccharide at 126. In some embodiments, the KI is added to the CTAB precipitated polysaccharide solution of the previous step to obtain a predefined KI percent for precipitating residual CTAB that is in the solution where the final KI concentration of between 26 and 29 mM is achieved. In some embodiments, the final KI concentration is approximately 27 mM. After KI concentration has been adjusted, the KI-precipitated polysaccharide solution is allowed to incubate for approximately 60-75 minutes at a predefined temperature, pH, and agitation rate. In some embodiments, the KI precipitation is carried out at a temperature between about 25° C. and about 33° C. In some embodiments, the agitation speed for the KI precipitation step is approximately 75 rpms. In some embodiments, the KI precipitation is carried out at a pH of between 6.6 and 7. After KI precipitation, the KI precipitated solution is centrifuged for about 30 minutes or more at a g-force of between 8800 to 9200 and a temperature between about 28° C. to about 32° C. resulting in the KI precipitated polysaccharide solution that is retained at 126 and where the pellet is discarded and the supernatant retained.

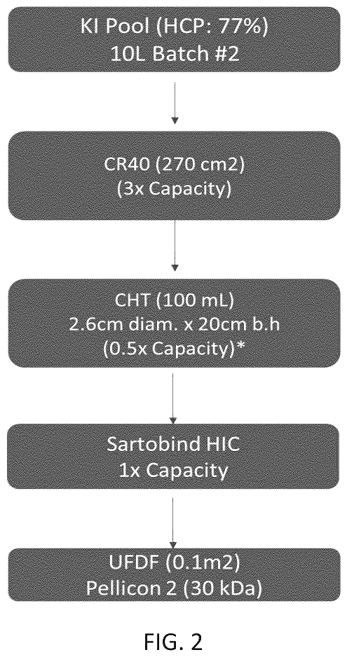

Staying with FIG. 1, following KI precipitation for the improved capsular polysaccharide purification, a carbon filter step at 128 may be applied to further remove impurities, resulting in a carbon-filtered polysaccharide. The carbon filter 128 comprises activated carbon and cellulose fibers (such as the Millistak+® from Millipore Sigma). Carbon filter 128 may be flushed with a phosphate buffer (Buffer C). In some embodiments, Buffer C may be 20 mM sodium phosphate and 350 mM NaCl at approximately pH 6.8. In some embodiments, the temperature for this step is between about 15° C. and about 25° C. As alluded to earlier, the stepwise yield at carbon filter 128 typically produced yields of between 20-30% for this step. Here, it was discovered that loading sample at greater than the suggested load capacities specified for a carbon filter (e.g., if the carbon filter capacity is X kg/m2, then 3× the amount of sample is loaded onto and flowed through the carbon filter) provided greater yields for the product but could also increase the amount of HCP coming through as well (which will be addressed in subsequent paragraphs of this disclosure). For example, in some instances where the carbon filter membrane capacity is rated for 50 kg/m² load capacity, a 3× load capacity of calculated sample (~150 kg sample) was applied to carbon filter 128. It is also possible to achieve the results of increasing product yield in this carbon filter step using a series of carbon filters where the sample sent through the one or more carbon filters is three times the load capacity for the calculated surface area of the one or more carbon filters. FIG. 2 shows excerpted steps of the overall capsular polysaccharide purification process in stepwise fashion.

Following the carbon filtration 128 and further filtering at filter 130, the carbon filtered polysaccharide can be run through two chromatography columns for further purification. At this stage, because of the increased load capacity at the carbon filter 128, while much greater yields of the desired polysaccharide were recovered, only a small amount of host cell proteins (HCPs) was removed from the carbon filtered polysaccharide at the carbon filter 128 step and further removal of HCPs is desired. ACHT column 132 may be employed followed by (e.g., in series with) a hydrophobic interaction (HIC) 134 chromatography. CHT 132 and HIC 134 can be equilibrated with a buffer that promotes binding of impurities (Buffer C). Buffer C may comprise Na₃PO₄, NaCl, and CTAB where its pH is about 7. In some embodiments, Buffer C is 20 mM Na3PO4, 400 mM NaCl, and 1% (w/w) CTAB with pH of approximately 6.8. One example of CHT 132 is a column having a diameter of 10 cm and a bed height of about 22 cm. The flow rate through CHT 132 can be approximately 150 cm/hr or a volumetric flow rate of about 12 L/hr. The pressure in the column should not exceed 4 bars.

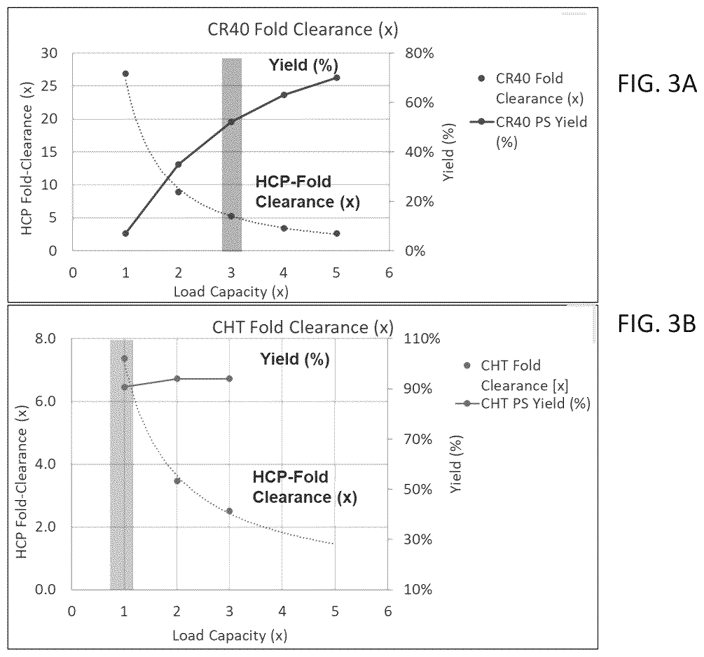

FIG. 3A shows graphical data for step at carbon filter 128 comparing the polysaccharide product yield as it relates to the amount of HCP clearance. As expected, as the load capacity for carbon filter 128 is pushed beyond its suggested capacity limits, the amount of polysaccharide product recovered increases but the downside of this is that the amount of HCP that is cleared by the filter is reduced significantly. While FIG. 3A shows that polysaccharide yields continue to increase at increasing load capacities, an increase of greater than three times the load capacity of polysaccharide samples hinders removal of HCP to desired manufacturing specifications at subsequent steps.

To address the greater amount of HCP that still remains in the carbon-filtered polysaccharide, the carbon-filtered polysaccharide is put there a CHT and HIC (hydrophobic interaction chromatography) column in series. FIG. 3B shows the results of the carbon-filtered polysaccharide being put through CHT 132 column. Here, it can seen that by loading the CHT 132 column at half the capacity for the size and/or volume of the column, the clearance at around 1× or slightly less than 1× the load capacity for the CHT column provided high HCP clearance. Increasing the load capacity for the CHT 132 column even slightly above 1× the load capacity in relation to the size of the CHT column showed a noticeable drop in HCP clearance. After the CHT (CHT polysaccharide) followed by HIC chromatography (HIC polysaccharide) steps, the HIC purified polysaccharide may be again run through a filter and the resulting solution collected. The filter can be a 0.2 micron filter.

Returning to FIG. 1 for the overall polysaccharide purification process, at the end of the HIC chromatography step 134, the HIC purified polysaccharide may be run through a second TFF step (shown as 136 in FIG. 1). This second TFF 136 step is performed at a lower molecular weight cut-off (MWCO) than the TFF 112 step, but still with polyethersulfone. In some embodiments, the tangential flow filtration (TFF) cassette comprises an ultrafiltration/diafiltration (UF/DF) arrangement. In some embodiments, the second TFF 136 step is performed at a MWCO of about 30 kDa. At this step, the HIC purified polysaccharide (which may or may not have been flowed through an additional filter) may be concentrated between 8× to 28× its original volume before being diafiltered (DF) against a buffer (e.g., Buffer C). Diafiltration can be performed in continuous mode. Post-diafiltration against buffer, the resulting solution containing the desired polysaccharide product can then subsequently be further diafiltered against purified water (e.g., Water for Injection (WFI)) and concentrated to a desired polysaccharide concentration. The polysaccharide resulting from the second TFF step may be further flowed through a final filtering step prior to collection and use in further downstream processes.

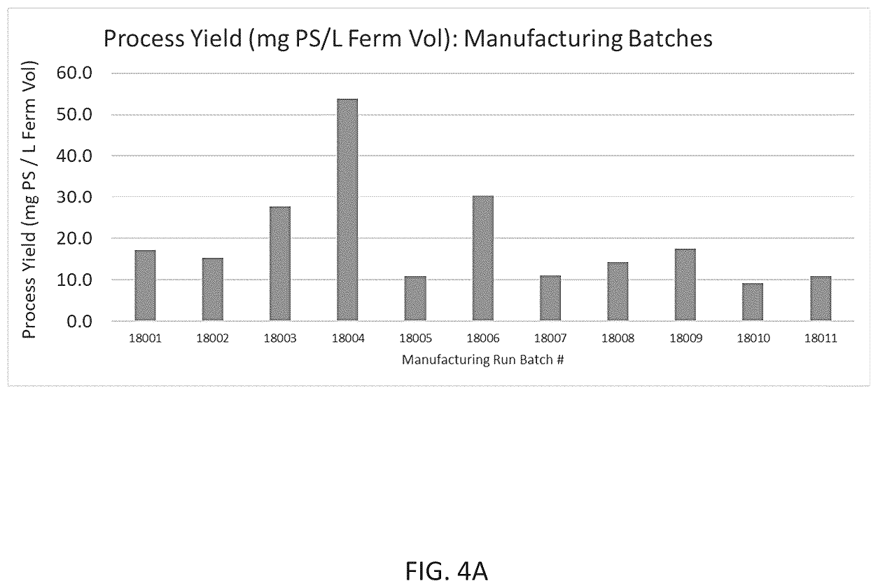

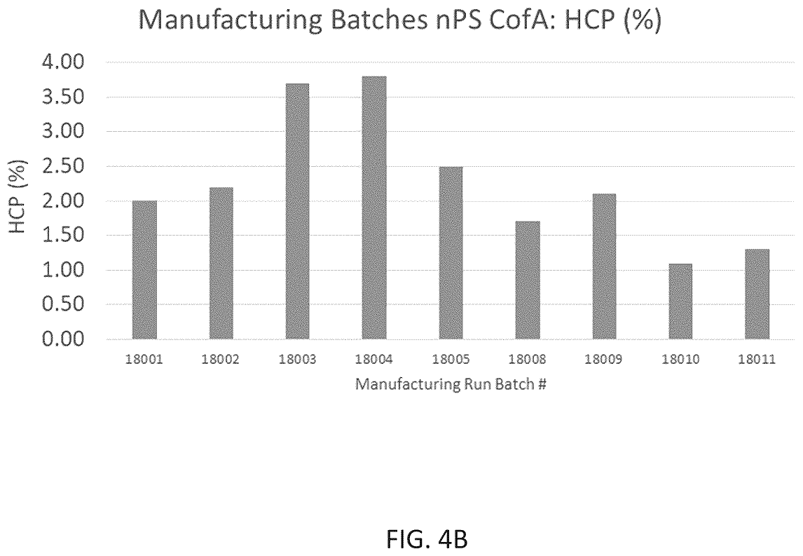

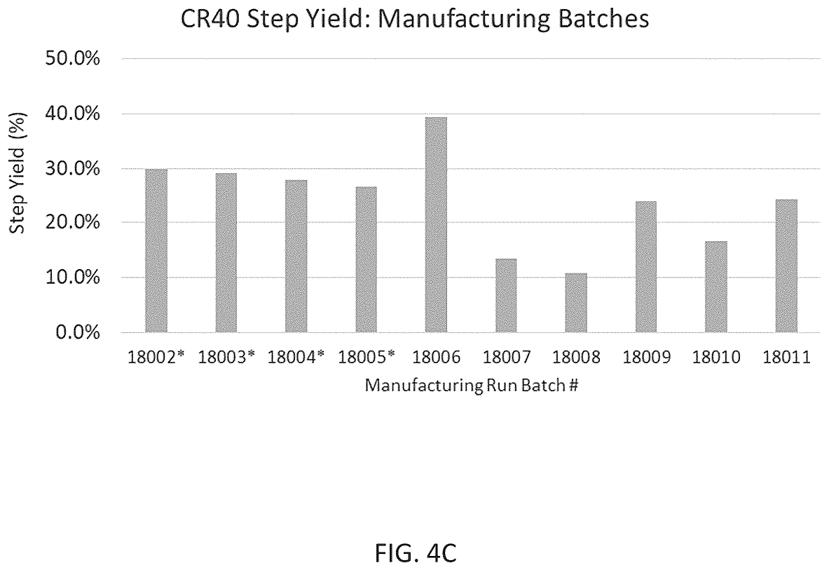

FIGS. 4A-4C show results from a standard purification process for a given polysaccharide. FIG. 4A shows eleven batches and their respective yields. The range of polysaccharide yield for these batches range from slightly under 10% yield to about 30% yield, with batch 18004 being an anomaly with a yield of 53%. For these same eleven batches, the percent of HCP ranges from a little over 1% to about 3.75% (FIG. 4B). And finally, as mentioned previously, the percent yield after the carbon filter step is shown for the eleven batches in FIG. 4C. On average the percent yield for the carbon filter step was from about 10% to about 30%, with batch 18006 having the highest percent yield of close to 40% as compared to the other batches examined.

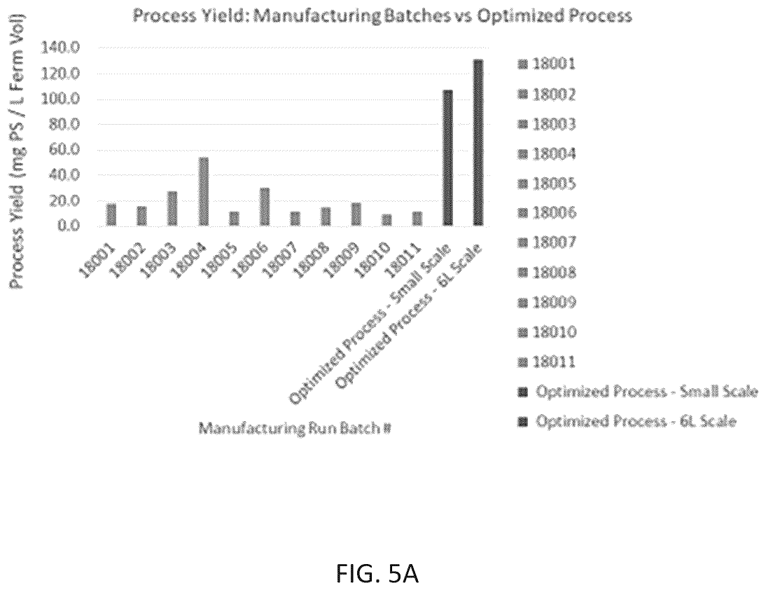

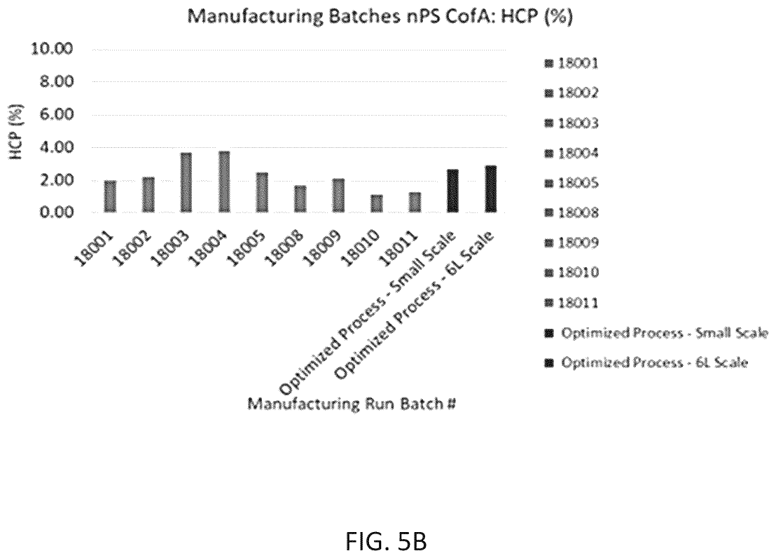

FIGS. 5A and 5B show a smaller batch and a larger batch of polysaccharides that were processed using the new conditions for the carbon filter and the subsequent CHT chromatography steps. FIG. 5A is a table comparing the overall process yields of prior polysaccharide batches with the improved purification conditions described above. The two batch runs using the improved process steps described herein (e.g., three times load capacity for the carbon filter and half the load capacity for CHT chromatography) has an overall yield of about 105 mg PS/L (polysaccharide/liter) for the small scale and about 130 mg PS/L compared to about 20 mg PS/L. This is at least five times increase in the overall polysaccharide yield. Importantly, the dramatic increase in overall polysaccharide yield was not accompanied by proportional increases in HCP in the sample. FIG. 5B shows a comparison of the percent HCP in prior polysaccharide manufacturing batch compared to the percent HCP in two batches using the improved purification process steps described here. As the two right-most bars show, the HCP in the samples processed with the improved purification process is comparable to that of prior run batches using what is common in the field for such types of purification.

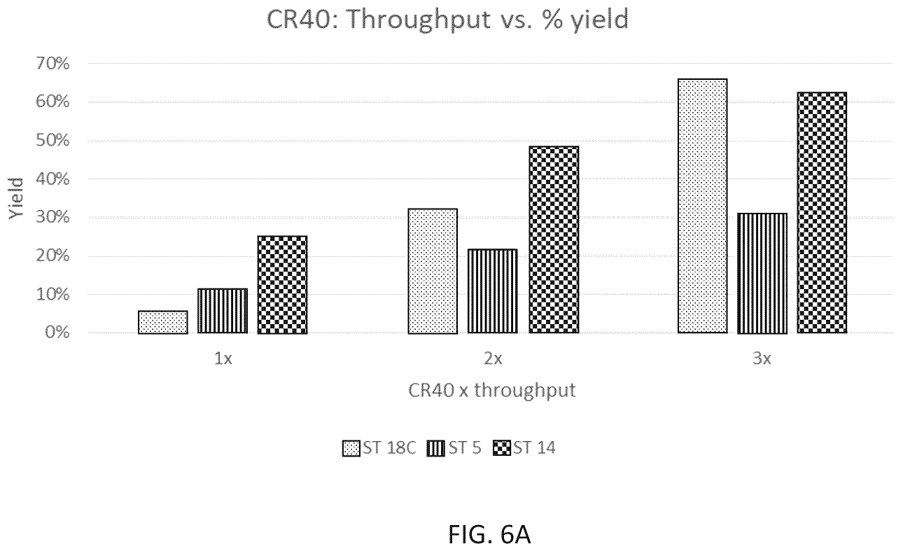

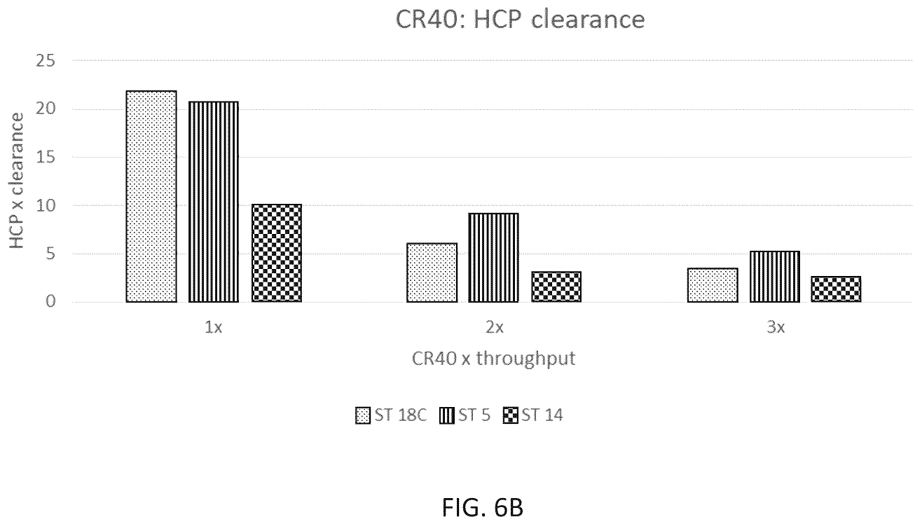

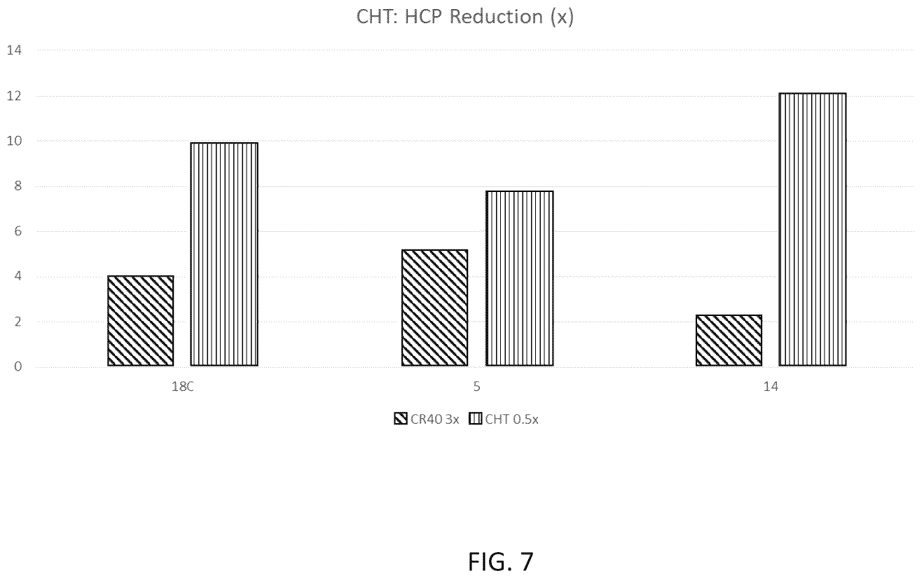

FIGS. 6A and 6B show additional data for the carbon filter 128 step for additional S. pneumoniae serotypes. FIG. 6A shows the percent yield for the carbon filter 128 step for three S. pneumoniae serotypes. As the graphs show, as more sample is loaded on to carbon filter 128, the amount of each serotype (% yield) increases for all three serotypes. FIG. 6B shows the HCP clearance at carbon filter 128 step for three S. pneumoniae serotypes. As expected with the increasing load onto carbon filter 128, clearance for HCP decreases significantly. Thus, the subsequent CHT 132 step plays a useful role under the conditions described to remove the additional HCP present from the prior carbon filter 128 step. FIG. 7 shows a comparison of HCP reduction for three S. pneumoniae serotypes where the CHT column is loaded at half capacity; this is compared to the amount of HCP reduction for at the carbon filter 128 step. As the data indicates, the reduction is greater at CHT at half capacity for all three S. pneumoniae serotypes.

Inventors envision that this improved process for purifying capsular polysaccharides can be used beyond polysaccharides of S. pneumoniae. In addition to improving serotype yields for S. pneumoniae, one skilled in the art would expect that this improved purification process may be useful for purifying capsular polysaccharides of meningitis, pertussis, and so forth.

A process for purifying a capsular polysaccharide (e.g., S. pneumoniae) cell lysate. The process described includes a carbon-filter step (128). In the carbon filter step (128), three times the load capacity of polysaccharide that the carbon filter is calculated for (based on surface area of the carbon filter) is loaded and run through the carbon filter. Furthermore, the purification process includes a CHT column chromatography step (132), where the carbon-filtered polysaccharide can be flowed through a CHT column where the CHT is loaded at 50% of the calculated load capacity for a CHT column of that size resulting in a CHT purified polysaccharide.

In some embodiments, after the batch fermentation process is completed and the cell batch has been put through a lysis treatment, the next process step comprises passing the capsular polysaccharide cell lysate through one or more depth filters. The one or more depth filters may be placed in series. In some embodiments, for an approximate 100-150 kg sample, an approximate combined filter area of 7 m²±10% may be used. In some embodiments, the depth filters may be agitated at between 40 to 60 rpm. In some embodiments the capsular polysaccharide cell lysate may be flowed through the one or more depth filters at a rate of less than 10 L/min and where the pressure does not exceed 2 bars.

The resulting depth-filtered polysaccharide may then be introduced into a TFF cassette for ultrafiltration and diafiltration (UF/DF). The TFF cassette includes a polyethersulfone membrane. In some embodiments, with the TFF cassette, the depth-filtered polysaccharide is concentrated by about ten folds. The concentrated UF concentrated polysaccharide may be allowed to circulate along a buffer (e.g Buffer J, where Buffer J can be about 25 mM sodium phosphate buffer and 444 mM NaCl pH 6.8) for a period of time (e.g., 15 minutes). The UF-concentrated polysaccharide may then go through a DF buffer exchange process. In some embodiments, the UF-concentrated polysaccharide is exchanged with about nine times the amount (by weight) of the UF-concentrated polysaccharide. In some embodiments, the TFF MWCO is about 100 kDa.

Prior to introducing the depth filtered-polysaccharide into a TFF (UF/DF) step, it can be put their a bioburden reduction filtration step. In some embodiments, the filter is approximately 0.45 microns and 0.2 micron. In some embodiments, the filtration flow rate is not more than 12 L/min and a pressure of no more than 2 bar.

The TFF-purified polysaccharide may be precipitated with a cationic surfactant resulting in a cationic surfactant-purified polysaccharide. The cationic surfactant can be CTAB. In some embodiments the TFF purified polysaccharide is treated with about 1% CTAB and about 0.4 M NaCl. In some embodiments, the cationic surfactant solution is added within a 15 minute time period and the TFF purified polysaccharide and the cationic surfactant combined solution is allowed to incubate for about an hour at between a temperature of 27° C. and 33° C. Prior to the CTAB precipitation, the TFF-purified polysaccharide may be put through a second depth filtration step. In some embodiments, the filtration pore size can be between 0.45 microns and 40 microns.

Following the CTAB precipitation step, a second precipitation step using KI can be employed to produce a KI precipitated polysaccharide. In some embodiments, the concentration of KI used is approximately between 26 mM and 29 mM. In some embodiments, the temperature for the KI precipitation is between 25° C. and 33° C. In some embodiments, the KI is added within a 15 minute window and the resulting solution is allowed to stir for about 60 minutes. In some embodiments the resulting solution is centrifuged for approximately 30 minutes at approximately 9000 g. In some instances, another depth filtration step may be employed.

Following the KI precipitation step, the KI precipitated polysaccharide can be put through a carbon filter resulting in a carbon-filtered polysaccharide as mentioned above where the load capacity for the carbon filter is about three times its calculated/suggested load capacity. In some embodiments, the maximum pressure employed is about 2 bars. In some embodiments, the feed flow rate is approximately 8 kg/10 min.

In some embodiments, the carbon-filtered polysaccharide is put through a CHT column where the load capacity of the CHT column is about 50% of the suggested/calculated load capacity for the CHT column (based on size and amount of resin). The CHT column has a column diameter of about 10 cm and a column bed height of approximately 22 cm. In some embodiments, for a suggested load capacity of 70 L fermentation volume per L of resin, only 30 L of carbon-filtered polysaccharide solution from the previous step is loaded on to the CHT column. In some embodiments, the CHT linear flow is approximately 150 cm/hr with a flow volume of approximately 12 L/hr. In some embodiments, the pressure is not above 4 bars. In some embodiments, the temperature is approximately between 15° C. and 25° C.

The CHT purified polysaccharide may then be placed through a HI column resulting in an HI purified polysaccharide. In some embodiments, the HI column may be about 36 cm in diameter and the column bed height is about 1 cm. In some embodiments, the linear flow rate is about 12 cm/hr and the volume flow is about 12 L/hr. In some embodiments, the pressure is not above 4 bars. In some embodiments, the temperature is approximately between 15° C. and 25° C.

The HI purified polysaccharide may then be placed through a second TFF cassette comprising another UF/DF arrangement. The second TFF wash steps are similar to those of the first TFF step. Here however, the MWCO for the membrane is lower than the previous TFF cassette. In some embodiments, the MWCO is about 30 kDa.

It should be noted that prior to implementing the purification steps mentioned above, the overall yield for three S. pneumoniae for which data is shown is between about 10-15%. The overall yield from the stepwise purification implementing the described carbon filter and CHT steps was about 30%.

### ENUMERATED EMBODIMENTS

Embodiment I-1. A process of purifying a capsular polysaccharide cell lysates to produce a purified polysaccharide, the process comprising the steps of:
(a) flowing a crude capsular polysaccharide cell lysate through a carbon filter wherein the carbon filter is loaded at a load capacity of three times the specified load capacity for the carbon filter; and
(b) flowing a resulting solution from step (a) through a ceramic hydroxyapatite column wherein the ceramic hydroxyapatite column is loaded at fifty percent load capacity.

Embodiment I-2. The process of embodiment I-1, further comprising passing the crude capsular polysaccharide cell lysate through a series of one or more depth filters prior to flowing the crude capsular polysaccharide cell lysate through the carbon filter.

Embodiment I-3. The process of embodiment I-1 or I-2 further comprising passing the crude capsular polysaccharide cell lysate through a first tangential flow filtration (TFF) system prior to flowing the crude capsular polysaccharide cell lysate through the carbon filter.

Embodiment I-4. The process of any one of embodiments I-1 to I-3 further comprising precipitating the crude capsular polysaccharide cell lysate with a solution of cetyltrimethylammonium bromide (CTAB) prior to flowing the crude capsular polysaccharide cell lysate through the carbon filter.

Embodiment I-5. The process of any one of embodiments I-1 to I-4 further comprising precipitating the crude capsular polysaccharide cell lysate with a solution of KI prior to flowing the crude capsular polysaccharide cell lysate through the carbon filter.

Embodiment I-6. The process of any one of embodiments I-1 to I-5 further comprising a step of using hydrophobic interaction chromatography (HIC) after flowing a resulting solution from step (a) through the ceramic hydroxyapatite (CHT) column.

Embodiment I-7. The process of embodiment I-1 further comprising a second TFF step after the CHT column.

Embodiment I-8. A process of purifying capsular polysaccharide cell lysates, the process comprising the steps of:
a) passing the capsular polysaccharide cell lysates through a first set of one or more depth filters placed in series resulting in a depth-filtered polysaccharide;
b) introducing the depth-filtered polysaccharide onto a first tangential flow filtration system resulting in a first TTF polysaccharide;
c) precipitating the first TFF polysaccharide with a cationic surfactant resulting in a cationic surfactant purified polysaccharide;
d) mixing the cationic surfactant purified polysaccharide with a solution of potassium iodide (KI) resulting in a KI purified polysaccharide that is in solution;
e) flowing the KI purified polysaccharide through a carbon filter, wherein a three times load capacity of the fourth purification step polysaccharide amount specified for the carbon filter is run through the carbon filter resulting in a carbon filter purified polysaccharide;
f) flowing the carbon filter purified polysaccharide through a ceramic hydroxyapatite (HI) membrane resulting in a ceramic hydroxyapatite purified polysaccharide.
g) flowing the ceramic hydroxyapatite purified polysaccharide through a hydrophobic interaction chromatography column resulting in a HI purified polysaccharide; and
h) flowing the HI purified polysaccharide through a second TFF process where the molecular weight cutoff (MWCO) is lower than that of the first TFF system to obtain a resulting final purified polysaccharide with an overall yield of at least 30%.

Embodiment I-9. The process of embodiment I-8, wherein the first set of depth filters of step (a) having a pore size ranging from about 0.2 microns to about 0.45 microns.

Embodiment I-10. The process of embodiment I-8 or I-9, wherein the tangential filtration flow of step (b) is performed with a molecular weight cut-off of 100 kDa.

Embodiment I-11. The process of any one of embodiments I-8 to I-10, wherein the cationic surfactant is CTAB.

Embodiment I-12. The process of any one of embodiments I-8 to I-11, wherein the cationic surfactant purified polysaccharide from the CTAB precipitation of step (c) is further put through a second depth filter, wherein the second depth filter comprises a matrix of cellulose and diatomaceous earth, wherein the second depth filter has pore sizes of about 0.4 microns to about 40 microns.

Embodiment I-13. The process of any one of embodiments I-8 to I-12, wherein the KI purified polysaccharide from the KI precipitation is centrifuged at a g-force range of 8800 to 9200.

Embodiment I-14. The process of embodiment I-13, wherein the KI precipitation is performed at 28-32° C.

Embodiment I-15. The process of any one of embodiments I-8 to I-14, wherein the ceramic hydroxyapatite column is used at a fifty percent load capacity for the ceramic hydroxyapatite column size.

Embodiment I-16. A process of purifying a capsular polysaccharide cell lysate to produce a purified polysaccharide, the process comprising the steps of:
a) passing the capsular polysaccharide cell lysates through at least one depth filter placed in series resulting in a depth filtered polysaccharide;
b) introducing the depth filtered polysaccharide onto a first tangential flow filtration (TFF) resulting in a first TTF purified polysaccharide;
c) precipitating the first TTF purified polysaccharide with a cationic surfactant resulting in a cationic surfactant purified polysaccharide;
d) mixing the cationic surfactant purified polysaccharide with a solution of potassium iodide (KI) resulting in a KI purified polysaccharide that is in solution;
e) flowing the KI purified polysaccharide through a carbon filter, wherein the KI purified polysaccharide is loaded at three times load capacity specified for the carbon filter resulting in a carbon filter purified polysaccharide;
f) flowing the carbon filter purified polysaccharide through a ceramic hydroxyapatite column resulting in a ceramic hydroxyapatite purified polysaccharide, wherein the ceramic hydroxyapatite column is used at a fifty percent load capacity specified for the ceramic hydroxyapatite's column size;
g) flowing the ceramic hydroxyapatite purified polysaccharide through a hydrophobic interaction (HI) chromatography column resulting in a HI purified polysaccharide; and
h) flowing the HI purified polysaccharide through a second TFF process to obtain a resulting final purified polysaccharide with an overall yield of at least 30%.

Embodiment I-17. The process of embodiment I-16, wherein the first set of depth filters of step (a) have a pore size ranging from about 0.2 microns to 0.45 microns.

Embodiment I-18. The process of embodiment I-16 or I-17, wherein the tangential filtration flow of step (b) is performed with a molecular weight cut-off of 100 kDa.

Embodiment I-19. The process of any one of embodiments I-16 to I-18, wherein the cationic surfactant is CTAB.

Embodiment I-20. The process of any one of embodiments I-16 to I-19, wherein the KI purified polysaccharide from the CTAB precipitation of step (c) is further put through a second depth filter, wherein the second depth filter comprises a matrix of cellulose and diatomaceous earth, and wherein the second depth filter has pore sizes of about 0.6 micron to about 40 microns.

Embodiment I-21. The process of any one of embodiments I-16 to I-20, wherein the KI purified polysaccharide is centrifuged at a g-force range of from 8800 to 9200.

Embodiment I-22. The process of embodiment I-21, wherein the process is performed at 28-32° C.

Embodiment I-23. The process of any one of embodiments I-16 to I-22, wherein the HI purified polysaccharide is passed through a second TFF process wherein the molecular weight cut off is 30 kDa.

### EXAMPLES

### Example 1: Process

Post fermentation and cell lysis, the following steps are performed under the following conditions:

### 1. Depth Filtration:

113 kg dH₂O filter pre-flush
30 kg-25 mM pH 6.8 Na₃PO₄ (444 nM NaCl) filter pre-flush
Area—Clarisolve/20 MS: 4.4 m2 (8×0.55m2)
Area Millistak/C0HC: 2.75 m2 (2×1.1 m2+0.55 m2)
agitation—50 (40-60) rpm
temperature RT
flow rate 9 (≤10) L/min
pressure maximum 2 bar
pressure blowout 0.3 bar
15 kg 25 mM pH 6.8 Na3PO4 (444 nM NaCl) filter post-flush

### 2. Bioburden Reduction Filtration 1 (Optional):

30 kg 1M NaOH wash
60 kg distilled water rinse
3 kg-25 mM pH 6.8 Na₃PO₄ (444 nM NaCl) filter pre-flush
[Sartopore Platinum, 0.45 μm+0.2 μm]
filter area 3 m² (3×1 m²)
temperature RT
agitation feed bag (magmix) 50 (40-60) rpm
10 kg Buffer J—filtration flow rate 10 (≤11.7) L/min
backpressure maximum 2 bar
0.8 kg-25 mM pH 6.8 Na₃PO₄ (444 nM NaCl) filter post-flush
post-flush: pressure blowout 0.3 bar

### 3. TFF (UF/DF) 1

Concentrate to 40 kg (UF)
60 kg 25 mM pH 6.8 Na₃PO₄ (444 nM NaCl) filter pre-flush
Diafiltration (DF) with 360 kg buffer (9×DF Volumes)
2. circulation for 15 min
circulation for 15 min after 3 and 6 DF-Volumes
   a) DF-Volumes/batch mode 1 (80 kg>40 kg)
   b) DF-Volumes/cont. mode 8
TFF cutoff 100 kDa
filter area 4 m²
Agitation—100 (90-110) rpm
Temperature 20 (15-25) ° C.
TMP (ultrafiltration) 0.55 (0.35-0.75) bar
TMP (diafiltration) 1.00 bar (0.80-1.20) bar
VCF 6.85-7.55
Cross flow rate 1600 (1500-1700) L/h
Circulate postflush (12.5 kg buffer J) for 15 min

### 4. Bioburden Reduction Filtration 2 (Optional):

12.5 kg-25 mM pH 6.8 Na₃PO₄ (444 nM NaCl) filter pre-flush
[Sartopore Platinum, 0.45 μm+0.2 μm]
Agitation—off
12.5 kg Buffer J/filter area 2 m² circulated
post-flush: temperature RT
filtration flow rate 9 (≤10) L/min
(BFD 2.6-4.) backpressure maximum 2 bar

### 5. CTAB Precipitation

0.4 M NaCl, 1% CTAB
Adjust Temp.: 30±3° C.
[CTAB] final: 1.0%
5.833 kg 30° C., CTAB addition CTAB addition: ≤15 min
Incubation time: 60-75 min
Temp.: 30±3° C.

### 6. Depth Filtration 2 (Optional)

Distilled water rinse—11 kg
5.5 kg-25 mM pH 6.8 Na₃PO₄ (444 nM NaCl) filter pre-flush
Filter—0.55 m²
Membrane cap.: 120 kg/m²
5.5 kg at RT, Conditioning Total area: 0.550 m²
Total cap.: 66.0 kg
14.9 kg at RT, Flush Max. pressure: 2.1 bar
Blow out RT Filter type: Clarisolve 40 MS
Feed flow: 8.0 kg/10 min
Flux: 87.3 L/m²/h
Temp.: ≤25° C.
Pressure: ≤2 bar

### 7. KI Precipitation

27.4 mM KI
[KI] final: 27.4 mM
8.131 kg 30° C. KI addition: ≤15 min
Incubation time
with stirring: 60-75 min
Incubation time: >60 min
centrifug. temp: 30±2° C.
g-force: 9,000 g
centrifug. time: 30 min
#of beakers: 92
#of centrifuges: 2
#of runs: 8
Temp.: 25-33° C.

### 8. Carbon Filtration

Distilled water rinse—35 kg
20 mM Na-Phosphate. pH 6.8, 350 mM NaCl—17 kg conditioning
20 mM Na-Phosphate. pH 6.8, 350 mM NaCl—24 kg flush
Adjust Temp.: 20±5° C.
35.2 kg RT [symbol] Rinse Membrane cap.: 50 kg/m2
17.6 kg RT [symbol] Conditioning Total area: 1.76 m2
Total cap.: 88.0 kg with load amount of about 260 kg
24.0 kg RT [symbol] Flush Max. pressure: 2.1 bar
Feed flow: 8.0 kg/10 min
Flux: 27.3 L/m2/h
Temp.: 20±5° C.
Pressure: ≤2 bar

### 9. Pre-CHT Filtration (Optional)

Conditioning and pH adjust with either NaOH or HCl for pH of between 6.6 and 7
filter capacity: ≥862 L/m2
total area: 0.119 m2
area/capsule: 0.520 m2
#capsules: 1
Membrane Type: PES
   pore size: 0.45+0.2 um
   Feed flow: 800.0 g/min
   Flux: 92.3 L/m2/h
   Temp.: 20±5° C.
   Pressure: ≤2 bar

### 10. CHT Chromatography

CV: 1.73 L
5.2 L RT [symbol] Pre-Equilibration, Cycles: 1
10.4 L RT [symbol] Equilibration 6 column diameter: 10.0 cm
102.9 L RT [symbol] Load bed height: 22.0 cm
5.2 L RT [symbol] Wash 3 linear flow: 150 cm/h
5.2 L RT [symbol] Strip (CHT) 3 vol. flow: 11.8 L/h
5.2 L RT [symbol] Strip (Sartobind) 3
Linear flow Load+Wash: 75 cm/h
1.0 M NaOH 6.9 L RT [symbol] CIP 2 4
5.2 L vertical-flow Load+Wash: 5.9 L/h
WP 3.5 L RT [symbol] Rinse 1 2 1-flow HETP: 60 cm/h
5.2 L RT [symbol] Restoration 3 v-flow HETP: 4.7 L/h
WP 3.5 L RT [symbol] Rinse 2 2 max. binding: 102.9 kg
binding capacity: 60 kg/L bed (but with a loading amount of 30 kg/L)
5.2 L RT [symbol] HETP buffer run 3 Max. pressure: 4.0 bar
I 0.07 L RT [symbol] HETP buffer spike 0.04
Pressure flow c.: 2.0 bar
H 3.5 L RT [symbol] HETP buffer run 2 Temp: 20±5° C.

### 1. HI Membrane Chromatography

Sartobind Phenyl
Membrane volume: 0.80 L
5.2 L RT [symbol] Pre-Equilibration Cycles: 1 0.4
10.4 L RT [symbol] Equilibration column diameter: 35.7 cm 0.9
102.9 L RT [symbol] Load bed height: 0.8 cm 17.5
5.2 L RT [symbol] Wash linear flow: 11.8 cm/h 0.9
5.2 L RT [symbol] Strip (CHT) vol. flow: 11.8 L/h 0.4
WP 5.2 L RT [symbol] Strip (Sartobind) 1-flow Load: 5.9 cm/h 0.4
1.0 M NaOH 6.9 L RT [symbol] CIP 2 v-flow Load: 5.9 L/h 1.6
WP 3.5 L RT [symbol] Rinse 1 Max. pressure: 4.0 bar 0.3
5.2 L RT [symbol] Restoration Pressure flow c.: 2.0 bar 0.4
WP 3.5 L RT [symbol] Rinse 2 Temp: 20±5° C.

### 2. Pre-TFF Filtration (Optional)

filter capacity: ≥862 L/m2
total area: 0.125 m2
area/capsule: 0.520 m2
#capsules: 1
Membrane Type: PES
   pore size: 0.45+0.2 μm
   Feed flow: 800.0 g/min
   Flux: 92.3 L/m²/h
   Temp.: 20±5° C.

### 3. TFF (UF/DF) 2

Membrane area: 2.00 m²
Distilled water, RT [symbol] Rinse #cassettes: 1
WFI, RT [symbol] Final Rinse
20 mM Na-Phosphate. pH 6.8, 350 mM NaCl, 30.0 kg RT [symbol] Conditioning Total area: 2.00 m²
initial VCF (volumetric concentration factor): 10.8
Ret initial: 10.00 kg
20 mM Na-Phosphate. pH 6.8, 350 mM NaCl, 40.0 kg RT [symbol] Diafiltration factor 1 (DF1 factor): 4.0
WFI-DF2 factor: 9.0
WFI 90.0 kg RT [symbol] DF 2 final VCF: 1.70
Ret final: 5.88 kg
Low Press. Recirc.: 15 min
WFI 1.50 kg RT [symbol] Flush 1 membrane charge: 24.6 g/m²
WFI 1.50 kg RT [symbol] Flush 2 Permeate Flux: 24 L/m²*h
Feed Flux: 320 L/m²*h
TMP (transmembrane pressure)-UF1: 0.5 bar
TMP-DFsUF2: 1.0 bar
Temp.: 20±5° C.

1. A process of purifying a capsular polysaccharide cell lysates to produce a purified polysaccharide, the process comprising the steps of:
(a) flowing a crude capsular polysaccharide cell lysate through a carbon filter wherein the carbon filter is loaded at a load capacity of three times the specified load capacity for the carbon filter; and
(b) flowing a resulting solution from step (a) through a ceramic hydroxyapatite column wherein the ceramic hydroxyapatite column is loaded at fifty percent load capacity.

2. The process of claim 1, further comprising passing the crude capsular polysaccharide cell lysate through a series of one or more depth filters prior to flowing the crude capsular polysaccharide cell lysate through the carbon filter.

3. The process of claims 1 or 2 further comprising passing the crude capsular polysaccharide cell lysate through a first tangential flow filtration (TFF) system prior to flowing the crude capsular polysaccharide cell lysate through the carbon filter.

4. The process of any one of claims 1-3 further comprising precipitating the crude capsular polysaccharide cell lysate with a solution of cetyltrimethylammonium bromide (CTAB) prior to flowing the crude capsular polysaccharide cell lysate through the carbon filter.

5. The process of any one of claims 1-4 further comprising precipitating the crude capsular polysaccharide cell lysate with a solution of KI prior to flowing the crude capsular polysaccharide cell lysate through the carbon filter.

6. The process of any one of claims 1-5 further comprising a step of using hydrophobic interaction chromatography (HIC) after flowing a resulting solution from step (a) through the ceramic hydroxyapatite (CHT) column.

7. The process of claim 1 further comprising a second TFF step after the CHT column. A process of purifying capsular polysaccharide cell lysates, the process comprising the steps of:
a) passing the capsular polysaccharide cell lysates through a first set of one or more depth filters placed in series resulting in a depth-filtered polysaccharide;
b) introducing the depth-filtered polysaccharide onto a first tangential flow filtration system resulting in a first TTF polysaccharide;
c) precipitating the first TFF polysaccharide with a cationic surfactant resulting in a cationic surfactant purified polysaccharide;
d) mixing the cationic surfactant purified polysaccharide with a solution of potassium iodide (KI) resulting in a KI purified polysaccharide that is in solution;
e) flowing the KI purified polysaccharide through a carbon filter, wherein a three times load capacity of the fourth purification step polysaccharide amount specified for the carbon filter is run through the carbon filter resulting in a carbon filter purified polysaccharide;
f) flowing the carbon filter purified polysaccharide through a ceramic hydroxyapatite (HI) membrane resulting in a ceramic hydroxyapatite purified polysaccharide.
g) flowing the ceramic hydroxyapatite purified polysaccharide through a hydrophobic interaction chromatography column resulting in a HI purified polysaccharide; and
h) flowing the HI purified polysaccharide through a second TFF process where the molecular weight cutoff (MWCO) is lower than that of the first TFF system to obtain a resulting final purified polysaccharide with an overall yield of at least 30%.

9. The process of claim 8, wherein the first set of depth filters of step (a) having a pore size ranging from about 0.2 microns to about 0.45 microns.

10. The process of claims 8 or 9, wherein the tangential filtration flow of step (b) is performed with a molecular weight cut-off of 100 kDa.

11. The process of any one of claims 8-10, wherein the cationic surfactant is CTAB.

12. The process of any one of claims 8-11, wherein the cationic surfactant purified polysaccharide from the CTAB precipitation of step (c) is further put through a second depth filter, wherein the second depth filter comprises a matrix of cellulose and diatomaceous earth, wherein the second depth filter has pore sizes of about 0.4 microns to about 40 microns.

13. The process of any one of claims 8-12, wherein the KI purified polysaccharide from the KI precipitation is centrifuged at a g-force range of 8800 to 9200.

14. The process of claim 13, wherein the KI precipitation is performed at 28-32° C.

15. The process of any one of claims 8-14, wherein the ceramic hydroxyapatite column is used at a fifty percent load capacity for the ceramic hydroxyapatite column size.

16. A process of purifying a capsular polysaccharide cell lysate to produce a purified polysaccharide, the process comprising the steps of:
a) passing the capsular polysaccharide cell lysates through at least one depth filter placed in series resulting in a depth filtered polysaccharide;
b) introducing the depth filtered polysaccharide onto a first tangential flow filtration (TFF) resulting in a first TTF purified polysaccharide;
c) precipitating the first TTF purified polysaccharide with a cationic surfactant resulting in a cationic surfactant purified polysaccharide;
d) mixing the cationic surfactant purified polysaccharide with a solution of potassium iodide (KI) resulting in a KI purified polysaccharide that is in solution;
e) flowing the KI purified polysaccharide through a carbon filter, wherein the KI purified polysaccharide is loaded at three times load capacity specified for the carbon filter resulting in a carbon filter purified polysaccharide;
f) flowing the carbon filter purified polysaccharide through a ceramic hydroxyapatite column resulting in a ceramic hydroxyapatite purified polysaccharide, wherein the ceramic hydroxyapatite column is used at a fifty percent load capacity specified for the ceramic hydroxyapatite's column size;
g) flowing the ceramic hydroxyapatite purified polysaccharide through a hydrophobic interaction (HI) chromatography column resulting in a HI purified polysaccharide; and
h) flowing the HI purified polysaccharide through a second TFF process to obtain a resulting final purified polysaccharide with an overall yield of at least 30%.

17. The process of claim 16, wherein the first set of depth filters of step (a) have a pore size ranging from about 0.2 microns to 0.45 microns.

18. The process of claims 16 or 17, wherein the tangential filtration flow of step (b) is performed with a molecular weight cut-off of 100 kDa.

19. The process of any one of claims 16-18, wherein the cationic surfactant is CTAB.

20. The process of any one of claims 16-19, wherein the KI purified polysaccharide from the CTAB precipitation of step (c) is further put through a second depth filter, wherein the second depth filter comprises a matrix of cellulose and diatomaceous earth, and wherein the second depth filter has pore sizes of about 0.6 micron to about 40 microns.

21. The process of any one of claims 16-20, wherein the KI purified polysaccharide is centrifuged at a g-force range of from 8800 to 9200.

22. The process of claim 21, wherein the process is performed at 28-32° C.

23. The process of any one of claims 16-22, wherein the HI purified polysaccharide is passed through a second TFF process wherein the molecular weight cut off is 30 kDa.

24. The process of claims 1, 8 or 16, wherein the final purified polysaccharide comprises less than 11% Host Cell Protein (HCP) in the product.
